(19) United States
(12) Patent Application Publication
(10) Pub. No.: US 2018/0056018 A1
(43) Pub. Date: Mar. 1, 2018

(54) Inhalation Systems, Devices and Methods

(71) Applicant: Cephalon, Inc., Frazer, PA (US)

(72) Inventors: Janice M.G. Canvin, Hookwood (GB); Marla D. Curran, Swedesboro, NJ (US); Robert B. Noble, Hamilton, OH (US); Saku JP Torvinen, Helsinki (FI); James Zangrilli, Philadelphia, PA (US)

(21) Appl. No.: 15/689,299

(22) Filed: Aug. 29, 2017

Related U.S. Application Data
(60) Provisional application No. 62/381,999, filed on Aug. 31, 2016.

(51) Int. Cl. A61M 16/00; A61M 15/00; A61M 16/14; G06F 19/00; C07K 16/28

(52) U.S. Cl. CPC A61M 16/024; A61M 15/009; A61M 16/14; G06F 19/3456; C07K 16/2866; A61M 2016/0018; G06F 19/363; A61M 2016/0027; A61M 2205/3592; A61M 2205/502; A61M 2230/40; A61M 2205/583; G06F 19/325; A61M 2202/064

(57) ABSTRACT

Digital systems, devices and methods for managing a drug therapy, such as an anti-IL5 mAb or anti-IL5 receptor mAb treatment regimen for patients suffering from severe asthma (e.g., eosinophilic asthma), are provided. The digital systems, devices and methods may include hardware and software for predicting patient responses to a long term anti-IL5 mAb or anti-IL5 receptor mAb treatment based on clinical data obtained following an initial treatment period.

### CROSS-REFERENCE TO RELATED APPLICATIONS

This application claims the benefit of U.S. Provisional Application No. 62/381,999, filed Aug. 31, 2016, the content of which is incorporated herein by reference in its entirety.

### TECHNICAL FIELD

Provided herein are digital systems, devices and methods for managing a drug therapy, such as an anti-IL5 mAb or anti-IL5 receptor mAb treatment regimen for patients suffering from severe asthma (e.g., eosinophilic asthma). The digital systems, devices and methods may be used to predict patient responses to a long term anti-IL5 mAb or anti-IL5 receptor mAb treatment based on baseline clinical data and clinical data obtained following an initial treatment period.

### BACKGROUND

A substantial number of patients with asthma are inadequately controlled despite the use of current guideline-based therapeutic strategies. Beyond the limitations imposed by persistent asthma symptoms and diminished quality of life, such patients remain at risk for asthma exacerbations and increased healthcare utilization, and often represent a substantial portion of the costs incurred within a healthcare system with respect to respiratory conditions.

Severe eosinophilic asthma (i.e., ≧400 cells/μl blood at screening) is a distinct and clinically meaningful asthma phenotype characterized by elevated sputum and blood eosinophils, and is associated with poor asthma control and increased risk of exacerbation. At present, patients suffering from severe eosinophilic asthma may be prescribed a long-term anti-IL5 mAb or anti-IL5 receptor mAb treatment regimen in an effort to address both current asthma impairment (e.g., lung function, asthma symptoms, and asthma-related quality of life) and the risk of future asthma exacerbations. However, the efficacy of such a regimen may not be apparent until the patients have undergone treatment for an extended period of time, such as for at least one year. As such, patients, insurers, healthcare providers, and/or healthcare benefit managers may incur significant costs associated with the treatment before it can be determined whether the treatment will be successful. Moreover, there may be an opportunity cost as well, as patients defer other potential treatment options while waiting to determine if the treatment regimen will ultimately be effective for them.

### SUMMARY

Disclosed herein are digital systems, devices and methods for managing a drug therapy, such as an anti-IL5 mAb or anti-IL5 receptor mAb treatment regimen for a patient suffering from severe asthma. The digital systems, devices and methods may employ hardware and software for processing clinical data and predicting the patient's response to the treatment regimen after the drug has been administered for only a portion of the treatment period typically considered necessary for evaluating the drug's efficacy. For example, in the case of a long-term anti-IL5 mAb or anti-IL5 receptor mAb treatment regimen involving reslizumab, a patient may undergo treatment for approximately one year (or 52 weeks) before it is determined whether the patient is responding to the treatment. The disclosed digital systems, devices and methods may shorten the evaluation period by enabling the patient and/or healthcare professional to predict whether the patient will be a likely responder at 52 weeks based on data gathered from an initial treatment interval, such as after the first 16 weeks of the therapy. In addition, the systems, devices and methods may enable assessment to be conducted outside of a clinical setting (e.g., at home), thereby avoiding costly and typically time consuming visits to a healthcare professional. Following the early assessment, if the patient is deemed to be a likely responder, the healthcare professional may decide to continue the drug therapy after the initial treatment window. Conversely, if the patient is deemed to be a likely non-responder, the healthcare professional may elect to discontinue or alter the treatment after the initial treatment window.

### BRIEF DESCRIPTION OF THE DRAWINGS

The summary, as well as the following detailed description, is further understood when read in conjunction with the appended drawings. For the purpose of illustrating the disclosed digital systems, devices and methods, there are shown in the drawings exemplary embodiments. However, the systems, devices and methods are not limited to the specific embodiments disclosed. In the drawings:

FIG. 1 is a diagram of a system for managing a drug therapy, such as an anti-IL5 mAb or anti-IL5 receptor mAb treatment regimen.

FIG. 2A is a perspective view of an exemplary inhaler that may be used to determine clinical data used in the management of a drug therapy.

FIG. 2B is a partial exploded view of the exemplary inhaler shown in FIG. 2A.

FIG. 2C is a system diagram of the exemplary inhaler shown in FIG. 2A.

FIG. 3 is a system diagram of an exemplary computing device that may be used to determine and process clinical data associated with the management of a drug therapy.

FIG. 4 is a simplex plot of a predicted response of an exemplary patient undergoing a drug therapy over an initial treatment period.

FIG. 5 is a simplex scatter plot of predicted responses of patients enrolled in two Phase 3 studies of a drug therapy for the treatment of severe eosinophilic asthma.

FIG. 6 is a simplex plot of the geometric demarcation of the three categorical outcomes illustrated in FIGS. 4 and 5.

### DETAILED DESCRIPTION OF ILLUSTRATIVE EMBODIMENTS

The disclosed digital systems, devices and methods may be understood more readily by reference to the following detailed description taken in connection with the accompanying figures, which form a part of this disclosure. It is to be understood that the disclosed systems, devices and methods are not limited to the specific embodiments described and/or shown herein, and that the terminology used herein is for the purpose of describing particular embodiments by way of example only and is not intended to be limiting of the claimed systems, methods, and devices.

Throughout this text, the descriptions refer to methods, as well as systems and devices for implementing the methods. Where the disclosure describes or claims a feature or embodiment associated with a method, such a feature or embodiment is equally applicable to the systems and devices implementing the methods. Likewise, where the disclosure describes or claims a feature or embodiment associated with a system or device implementing a method, such a feature or embodiment is equally applicable to the method.

When values are expressed as approximations, by use of the antecedent “about”, it will be understood that the particular value forms another embodiment. Reference to a particular numerical value includes at least that particular value, unless the context clearly dictates otherwise.

It is to be appreciated that certain features of the disclosed systems, devices and methods that may be, for clarity, described herein in the context of separate embodiments, may also be provided in combination in a single embodiment. Conversely, various features of the disclosed systems, devices and methods that may be, for brevity, described in the context of a single embodiment, may also be provided separately or in any sub-combination.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include the plural.

The term “about” when used in reference to numerical values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

The following abbreviations are used throughout the disclosure: Asthma Control Questionnaire (ACID); Asthma Quality of Life Questionnaire (AQLQ); clinical asthma exacerbation (CAE); confidence interval (CI); forced expiratory volume in 1 second (FEV₁); Global Initiative for Asthma (GINA); inhaled corticosteroid (ICS); and minimally clinically important difference (MCID).

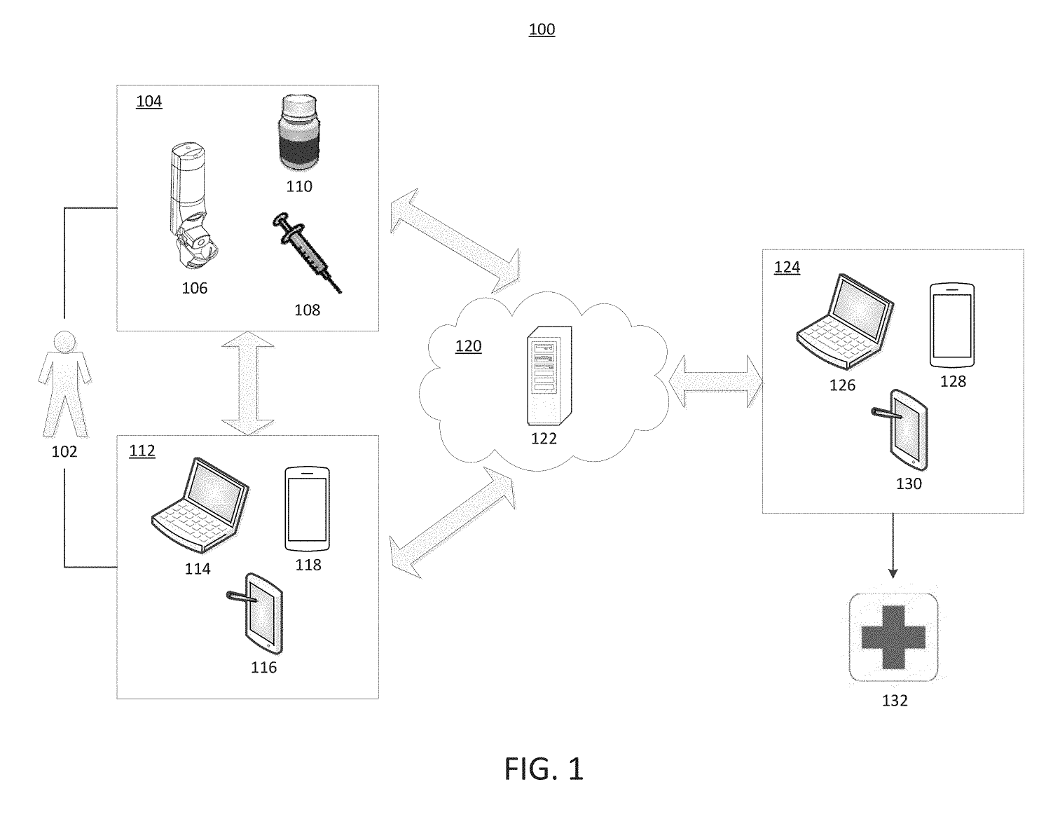

FIG. 1 illustrates an exemplary system 100 for managing a drug therapy, such as an anti-IL5 mAb or anti-IL5 receptor mAb treatment regimen for patients suffering from severe asthma or having eosinophilic asthma. In one embodiment, the anti-IL5 mAb or anti-IL5 receptor mAb treatment regimen may include reslizumab, mepolizumab, or benralizumab. Treatment regimens involving reslizumab, mepolizumab, or benralizumab may be administered parenterally, such as via a subcutaneous injection or intravenous infusion.

The treatment regimen may further include administering reslizumab, mepolizumab, or benralizumab for an initial treatment period, which may be approximately 4 weeks, 8 weeks, 12 weeks, 16 weeks or 20 weeks. After the initial treatment period, one or more components of the system 100 may be used to predict a patient's response to a long-term reslizumab, mepolizumab, or benralizumab treatment regimen, which may be approximately 36 weeks, 40 weeks, 44 weeks, 48 weeks, or 52 weeks. In a preferred embodiment, reslizumab may be administered to a patient over an initial treatment period of 16 weeks and the patient's likely response to reslizumab after 52 weeks of treatment may be predicted based on clinical data collected and obtained from the initial treatment period.

As shown in FIG. 1, the system 100 may include a patient 102, medications/medical devices 104 and a healthcare professional(s) 132, which may include doctors, nurses, pharmacists, insurers, benefit managers, drug manufacturers, drug providers, and the like. The patient 102 may be using one or more medications/medical devices 104 at the direction of the healthcare professional 132 for the treatment of a medical condition, such as asthma. The medications/medical devices 104 may include any type of pharmaceutical drug in any suitable dosage form, such as pills, tablets, capsules, liquids, powders, lotions, and the like. The medications/medical devices 104 may also include apparatuses or devices for storing the pharmaceutical drug and/or administering the drug to the patient 102. Exemplary apparatuses of the medications/medical devices 114 include an inhaler 106, an injector 108, and a pill container 110, although other apparatuses (such as patches and pumps) may be used as well. In addition to having internal components for storing, administering and/or delivering the pharmaceutical drug, the inhaler 106, the injector 108 and the pill container 110 may include electrical components (not shown), like one or more sensors for determining when and how the devices are being used and a communications interface for communicating such information to an external computing device.

The system 100 may further include computing devices 112 used by the patient 102 and computing devices 124 used by the healthcare professional 132. Exemplary computing devices 112 include a computer/laptop 114, a tablet 116, and/or a smartphone 118. Exemplary computing devices 124 include a computer/laptop 126, a tablet 130 and a smartphone 128. Each of the computing devices 112, 124 may also include a communications interface (not shown) for communicating information with an external device. For example, the communications interface of the computing devices 112 may be used to receive data from, and/or send data to, the inhaler 106, the injector 108, and/or the pill container 110. Each of the computing devices 112, 124 may also include a user interface for receiving and/or displaying information.

It will be appreciated that the medications/medical devices 114 and the computing devices 112 are generally associated with the patient 102 and are often portable. As such, the devices 112, 114 may be used outside of a medical or clinical setting, such as in a home or office.

The system 100 may further include a wireless network 120, which may include a radio access network (RAN), a core network, a public switched telephone network (PSTN), and/or the Internet. The wireless network may utilize equipment 122 to enable data to be exchanged wirelessly between multiple parties and entities. The equipment 122 may include base stations, servers, gateways, controllers, routers, databases and the like. The equipment 122 may employ any suitable networking and wireless technologies (e.g., CDMA, TDMA, FDMA, OFDMA, SC-FDMA, etc.) as part of the implementation of a cellular system, like wideband CDMA (WCDMA), long term evolution (LTE) and/or LTE-advanced (LTE-A). The wireless network 120 may be in wireless communication with the medications/medical devices 104 and/or the computing devices 112, 124. As such, the wireless network 120 may facilitate the exchange of information between the patient 102 and the healthcare professional 132.

The system 100 may further include a therapy software module (not shown) for managing a particular treatment regimen of the patient 102. The therapy software module may be implemented on any one of the medications/medical devices 104, the computing devices 112, 124 and/or the wireless network 120. Alternatively, portions of the therapy software module may reside on any of the foregoing nodes. The therapy software module may receive and process data from the medications/medical devices 104, the computing devices 112, 124 and/or the wireless network 120 to determine whether the patient 102 is likely to respond the treatment regimen.

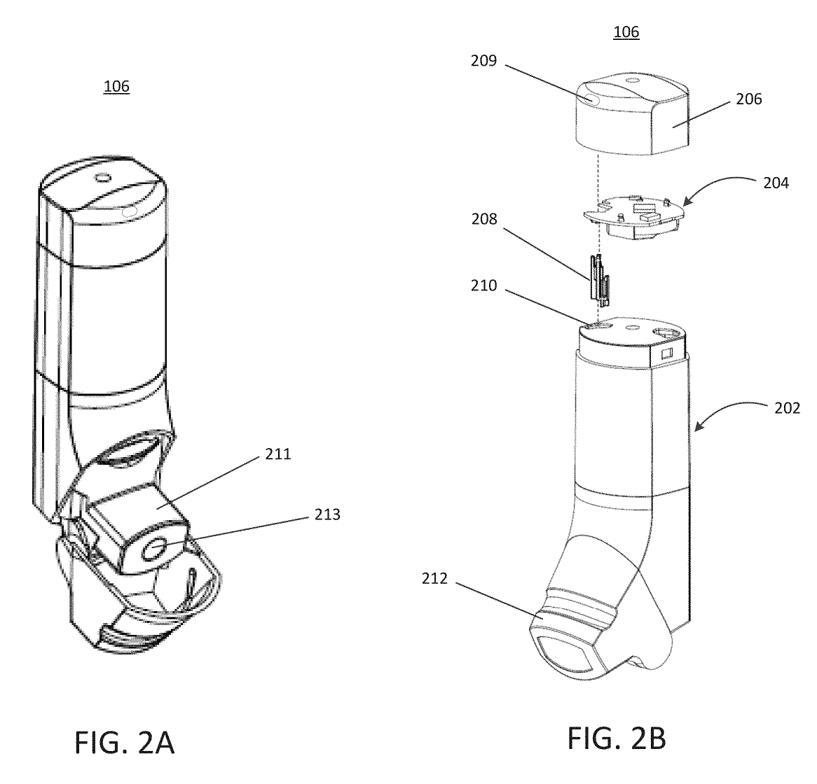

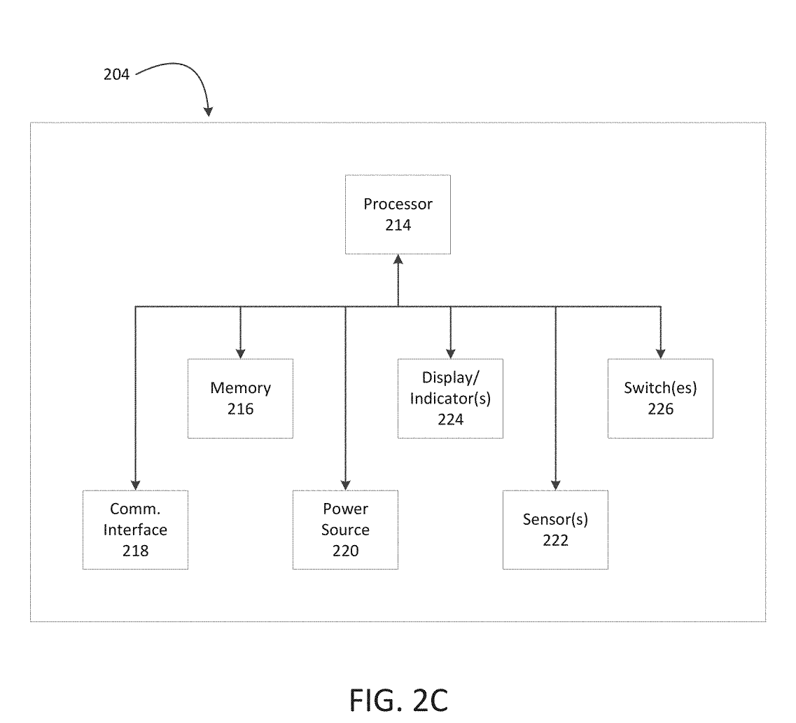

FIGS. 2A and 2B depict a perspective view and a partial exploded view of the exemplary inhaler 106, respectively. The inhaler 106 may be a metered dose inhaler (MDI) or a dry powder inhaler, such as the inhaler described in U.S. application Ser. No. 62/424,306, which is incorporated by reference herein in its entirety. The inhaler 106 may be configured to store and dispense any type of pharmaceutical drug for treating a respiratory condition, such as asthma or chronic obstructive pulmonary disease (COPD). In one embodiment, the inhaler 106 may dispense a corticosteroid.

The inhaler 106 may include a main body 202, which may house a reservoir (not shown) for storing a pharmaceutical drug and a delivery mechanism (not shown) for dispensing the pharmaceutical drug through a flow pathway 213 within a mouthpiece 211. The inhaler 106 may also include an electronics module 204 that is secured and/or housed within a cap 206, which may be attachably coupled to the top portion of the main body 202. The inhaler 106 may further include a slider 208, a portion of which may be configured to extend through an opening 210 on the main body 202. An opposing end of the slider 208 may extend into the cap 206.

The cap 206 may include a lens 209, which may be clear or translucent, thereby permitting light to pass through the cap 206. As will be further discussed below, the light may be generated or emitted from a light source, such as an LED, disposed on the electronics module 204. The light source may be used to provide indications or notifications to the patient 102 regarding the use of the inhaler 106.

The inhaler 106 may include a mouthpiece cover 212, which may be mechanically coupled to the slider 208 such that the opening of the mouthpiece cover 212 may cause the slider 208 to move along a vertical axis. As the slider 208 moves vertically (either up or down), the slider 208 may make contact with a switch (not shown) on the electronics module 204, thereby causing the electronics module 204 to transition to an active or sensing state.

FIG. 2C illustrates an exemplary system diagram of the electronics module 204. The electronics module 204 may include a processor 214, a memory 216, a communication interface 218, a power source 220, one or more sensors 222, one or more displays/indicators 224 and/or one or more switches 226. Each of the forgoing components may be physically distinct components within the electronics module 204. Alternatively, some or all of the components may be part of a single package. For example the processor 214, the memory 216 and the communication interface 218 may be part of a wireless chipset.

The processor 214 may access information from, and store data in the memory 216, which may include any type of suitable memory, such as non-removable memory and/or removable memory. The non-removable memory may include random-access memory (RAM), read-only memory (ROM), a hard disk, or any other type of memory storage device. The removable memory may include a subscriber identity module (SIM) card, a memory stick, a secure digital (SD) memory card, and the like. The processor 214 may also access data from, and store data in, memory that is not physically located within the electronics module 204, such as data located on a server or a smartphone.

The sensor(s) 222 may include one or more pressure sensors, such as a barometric pressure sensor (e.g., an atmospheric pressure sensor), a differential pressure sensor, an absolute pressure sensor, and/or the like. The sensor 222 may employ microelectromechanical systems (MEMS) and/or nanoelectromechanical systems (NEMS) technology. The sensor 222 may be configured to provide an instantaneous pressure reading and/or aggregated pressure readings over time. The sensor 222 may be disposed on the electronics module 204 and, as such, may be housed within the cap 206 on the top of the main body 202 of the inhaler 106. Thus, as illustrated in FIGS. 2A and 2B, it will be appreciated that the sensor 222 may detect pressure changes proximate to electronics module 204, i.e., pressure changes outside of the flow pathway 213 that is used to deliver a pharmaceutical drug to the patient 102.

The processor 214 may receive signals corresponding to pressure measurements from the sensor 222. The processor 214 may calculate or determine one or more airflow metrics using the signals received from the sensor 222. The airflow metrics may be indicative of a profile of airflow through the flow pathway 213 of the inhaler 106. For example, if the sensor 222 records a change in pressure of 0.3 kilopascals (kPA), the processor 214 may determine that the change corresponds to an airflow rate of approximately 45 liters per minute (Lpm) through the flow pathway 213. It will be appreciated that the conversion of pressure measurements to airflow rates may depend on the size, shape and design of the inhaler 106 and its associated components.

The airflow metrics may include one or more of an average flow of an inhalation/exhalation, a peak flow of an inhalation/exhalation (e.g., a maximum inhalation received), a volume of an inhalation/exhalation, a time to peak of an inhalation/exhalation, and/or the duration of an inhalation/exhalation. The airflow metrics may also be indicative of the direction of flow through the flow pathway 213. That is, a negative change in pressure may correspond to an inhalation from the mouthpiece while a positive change in pressure may correspond to an exhalation into the mouthpiece. The one or more pressure measurements and/or airflow metrics may be time-stamped and stored in the memory 216.

The processor 214 may compare signals received from the sensor 222 and/or the determined airflow metrics to one or more thresholds or ranges as part of an assessment of how the inhaler 106 is being used and whether the use is likely to result in the delivery of a full dose of medication. For example, where the determined airflow metric corresponds to an inhalation with an airflow rate below a particular threshold, the processor 214 may determine that there has been no inhalation or an insufficient inhalation from the mouthpiece 211. If the determined airflow metric corresponds to an inhalation with an airflow rate within a particular range, the processor 214 may determine that the inhalation is “good”, or likely to result in a full dose of medication being delivered. As noted above, the electronics module 204 may include display/indicators 224, such as an LED, for providing feedback to users regarding the use of the inhaler 106. Thus, in one example, an LED may illuminate or change color if the airflow metrics correspond to a good inhalation or to no inhalation. The illumination or change in color may be observed by the patient 102 via the lens 209 on the cap 206.

The processor 214 may also use the data from the sensor 222 and/or the determined airflow metrics to determine a measure of the lung function or lung health of the patient 102, as described in U.S. application Ser. No. 14/802,675, which is incorporated by reference herein in its entirety. In particular, the processor 214 may use a peak pressure detected by the sensor 222 to determine a maximum flow rate of an inhalation from the inhaler 106. The processor 214 may also use a series of pressure measurements from the sensor 222 to determine the volume of the inhalation. The processor 214 may then correlate the maximum flow rate with a peak inspiratory flow (PIF) and/or a peak expiratory flow (PEF) of an inhalation cycle and correlate the inhaled volume with FEV₁. It will be appreciated that the airflow metrics and/or measures of lung function can be processed by processors external to the inhaler 106. For example, data from the sensor 222 may be communicated to the computing devices 112, 124 and/or the wireless network 120 for further processing.

The communication interface 218 of the electronics module 204 may include a transmitter and/or receiver (e.g., a transceiver), as well as additional circuity such as an antenna. For example, the communication interface 218 may include an IEEE 802.11 chipset, a Bluetooth chipset (e.g., a Bluetooth Low Energy chipset), a ZigBee chipset, a Thread chipset, etc. As such, the electronics module 204 may wirelessly provide data such as pressure measurements, airflow metrics and/or other conditions related to usage of the inhaler 106 to an external device, such as one of the computing devices 112, 124. The computing devices 112, 124 may include software for processing the received information and for providing compliance and adherence feedback to the patient 102 and/or the healthcare professional 132 via a graphical user interface (GUI). In other embodiments, the communication interface 218 may include a cellular chipset, which may enable the electronics module 204 to communicate directly with the wireless network 120.

The power source 220 may provide power to the components of the electronics module 204. In one embodiment, the power source 220 may be a coin cell battery, for example, and may be rechargeable or non-rechargeable

The switch 226 may be used to “wake” the electronics module from an inactive or “sleep” state. For example, as noted above, the opening of the mouthpiece cover 212 may cause the slider 208 to move vertically within the cap 206. This vertical movement may cause the slider 208 to engage or disengage with the switch 226, thereby causing the electronics module to transition to an active state, which may permit the sensor 222 to begin taking pressure measurements and the processor 214 to begin computing airflow metrics.

While the system diagram of FIG. 2C has been described in the context of the inhaler 106, the disclosed components and accompanying description of the electronics module 204 are equally applicable to other types of devices among the medications/medical devices 104 shown in FIG. 1. For example, the electronics module 204 may also be incorporated into the injector 108 and/or the pill container 110. In such an embodiment, the electronics module 204 may track when and/or how a pharmaceutical drug was dispensed from the injector 108 or when a pill or tablet was removed from the pill container 110. Such information may be captured and communicated to one or more of the computing devices 112, 124.

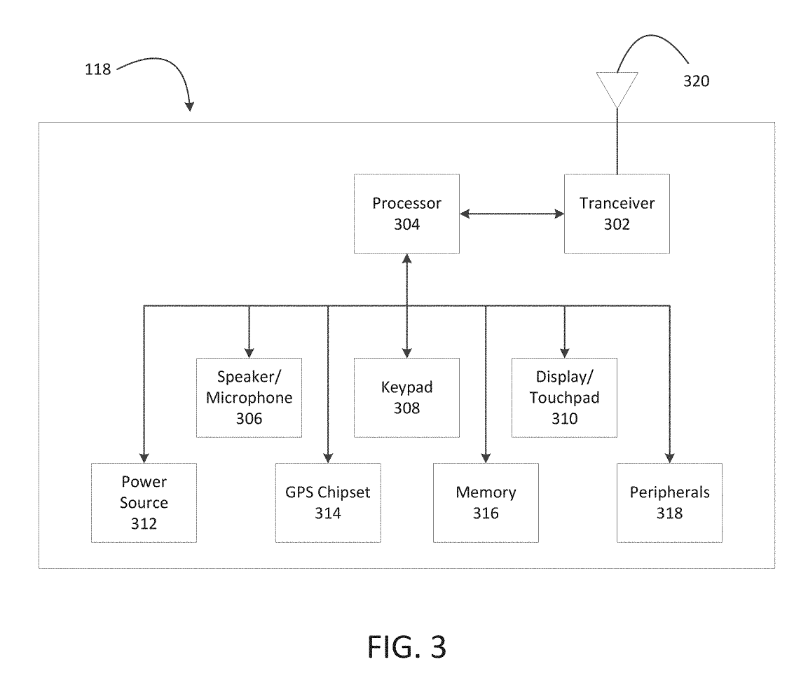

FIG. 3 illustrates an exemplary system diagram of the smartphone 118 of the computing device 112. As noted above, the smartphone 118 may be used by the patient 102 to communicate with one or more of the medications/medical devices 104 and/or the wireless network 120. The smartphone 118 may include a transceiver 302, a processor 304, an antenna 320, a speaker/microphone 306, a keypad 308, a display/touchpad 310, memory 316, a power source 312, a global positioning system (GPS) chipset 314, and peripherals 318. It will be appreciated that the smartphone 118 may include any sub-combination of the foregoing elements while remaining consistent with an embodiment.

The processor 304 may be a general purpose processor, a special purpose processor, a conventional processor, a digital signal processor (DSP), a microprocessor, one or more microprocessors in association with a DSP core, a controller, a microcontroller, an application specific integrated circuit (ASIC), a field programmable gate array (FPGA) circuit, an integrated circuit (IC), a state machine, and the like. The processor 304 may perform signal coding, data processing, power control, input/output processing, and/or any other functionality that enables the smartphone 118 to operate in a wireless environment. The processor 304 may be coupled to the transceiver 302, which may be coupled to the antenna 320. While FIG. 3 depicts the processor 304 and the transceiver 302 as separate components, the processor 304 and the transceiver 302 may be integrated together in a single electronics package or chip.

The antenna 320 may be configured to transmit wireless signals to, or receive wireless signals from, a base station in the wireless network over an air interface. For example, the antenna 320 may be configured to transmit and/or receive RF signals. In other embodiments, the antenna 320 may be an emitter/detector configured to transmit and/or receive IR, UV, visible light signals, and/or a combination of RF and light signals. While the antenna 320 is depicted in FIG. 3 as a single element, the smartphone 118 may include more than one antenna to utilize multiple input, multiple output (MIMO) technology.

The transceiver 302 may be configured to modulate/demodulate wireless signals transmitted/received by the antenna 320. In one or more embodiments, the smartphone 118 may have multi-mode capabilities. As such, the smartphone 118 may include multiple transceivers 302 for enabling the smartphone 118 to communicate over various radio access technologies (RATs), such as Bluetooth®, IEEE 802.11, 3G, 4G, and LTE, for example.

The processor 304 of the smartphone 118 may be coupled to, and may receive user input data from, the speaker/microphone 306, the keypad 308, and/or the display/touchpad 310, which may include any suitable type of display such as a liquid crystal display (LCD) display unit or an organic light-emitting diode (OLED) display unit. The processor 304 may also output user data to the speaker/microphone 306, the keypad 308, and/or the display/touchpad 310. In addition, the processor 304 may access information from, and store data in, in the memory 316, which may include non-removable memory and/or the removable memory. The non-removable memory may include random-access memory (RAM), read-only memory (ROM), a hard disk, or any other type of memory storage device. The removable memory may include a subscriber identity module (SIM) card, a memory stick, a secure digital (SD) memory card, and the like. In other embodiments, the processor 304 may access information from, and store data in, memory that is not physically located in the smartphone 118, such as data located on a server or a home computer (not shown).

The processor 304 may receive power from the power source 312, and may be configured to distribute and/or control the power to the other components in the smartphone 118. The power source 312 may be any suitable device or component for powering the smartphone 118. For example, the power source 312 may include one or more dry cell batteries, such as nickel-cadmium (NiCd), nickel-zinc (NiZn), nickel metal hydride (NiMH), lithium-ion (Li-ion), and the like. Alternatively, or in addition to dry cell batteries, the power source 312 may include solar cells and/or fuel cells.

The processor 304 may also be coupled to the GPS chipset 314, which may be configured to provide location information (e.g., longitude and latitude) regarding the current location of the smartphone 118. In addition to, or in lieu of, the information from the GPS chipset 314, the smartphone 118 may receive location information from the wireless network via the air interface 116 and/or determine its location based on the timing of the signals being received from two or more nearby base stations within the wireless network 120.

The processor 304 may further be coupled to other peripherals 318, which may include one or more software and/or hardware modules that provide additional features, functionality and/or wired or wireless connectivity. For example, the peripherals 318 may include an accelerometer, an e-compass, a satellite transceiver, a digital camera (for photographs or video), a universal serial bus (USB) port, a vibration device, a television transceiver, a hands free headset, a digital music player, a media player, a video game player module, an Internet browser, a mobile app and the like. In addition to any of the foregoing, the peripherals 318 may include the therapy software module for managing treatment regimens associated with the patient 102.

While the system diagram of FIG. 3 has been described in the context of the smartphone 118, it will be appreciated that the disclosed components and accompanying description are equally applicable to other devices of the computing devices 112, 124, such as the computer/laptop 114, 126, the tablet 116, 130 and/or the smartphone 128.

As noted above, the system 100 may include the therapy software module for managing a particular treatment regimen of the patient 102. The therapy software module may be stored in the memory 216 of the electronics module 204, the memory 316 of the computing devices 112, 124 and/or a server of the wireless network 120. The therapy software module may be executed by the processor 214 of the electronics module 204, the processor 304 of the computing devices 112, 124 and/or the server of the wireless network 120.

In one embodiment, the patient 102 may be suffering from severe asthma and/or have eosinophilic asthma and may be prescribed an anti-IL5 mAb treatment regimen, such as reslizumab, by the healthcare professional 132. Reslizumab may be administered via the injector 108, which may be a needle or auto-injector. Prior to the commencement of the treatment regimen, the healthcare professional 132 may collect certain baseline clinical data from the patient 102.

For example, the healthcare professional 132 may determine the number of CAEs experienced by the patient 102 over the previous year and enter such information into one or more of the computing devices 124 via the keypad 308 and/or the display/touchpad 310. The healthcare professional 132 may also determine the current FEV₁ of the patient 102 through the use of a spirometer and enter the lung function metric into one or more of the computing devices 124. Alternatively, FEV₁ may also be measured through the use of the inhaler 106 by the patient 102. In particular, specific FEV₁ values may be determined by the inhaler 106 and/or any of the computing devices 112, 124 with access to the data from the sensor 222 in the inhaler 106. The patient 102 may also complete an ACQ and an AQLQ via one of the computing devices 112, 124. The ACQ may include ACQ6 or ACQ7. The ACQ6 may include a questionnaire of six questions completed by the patient 102 that quantitatively measures both the adequacy of asthma control and change in asthma control, which may occur either spontaneously or as a result of medical treatment. The ACQ7 may include ACQ6 plus one additional question answered by the healthcare professional. The AQLQ may be a type of questionnaire completed by the patient 102 that quantitatively measures physical, emotional, social, and/or occupational problems or issues that may be experienced by patients suffering from asthma. Each of the foregoing data points (e.g., the number of CAEs, FEV₁ measurements, and/or the results of the ACQ and the AQLQ) may be collected and processed by the therapy software module and stored as a baseline score in the computing devices 112, 124 and/or the wireless network 120.

After the patient 102 has undergone treatment for an initial period of time, additional clinical data points may be collected. In one embodiment, the initial treatment period may be 16 weeks. At that time, the healthcare professional 132 may determine the number of CAEs experienced by the patient 102 over the initial treatment period along with the new FEV₁ score (via the spirometer and/or the inhaler 106). In addition, the patient 102 may again complete the ACQ and AQLQ. Like the baseline scores, the clinical data points collected at or after 16 weeks may be processed by the therapy software module and stored in the computing devices 112, 124 and/or the wireless network 120. The therapy software module may further analyze both sets of data to predict whether the patient 102 is likely to be a responder or a non-responder after undergoing long-term treatment, such as treatment for 52 weeks. If the therapy software module determines that the patient 102 is likely to be a responder, the therapy software module may generate a responder notification, which may result in the healthcare professional 132 continuing to prescribe the reslizumab treatment for the patient 102. If the therapy software module determines that the patient 102 is likely to be a non-responder after undergoing treatment for 52 weeks, the therapy software module may generate via a non-responder notification, which may result in the healthcare professional 132 discontinuing or altering the reslizumab treatment. The responder and non-responder notifications may be generated via the display/indicator(s) 224 of the electronics module 204 and/or via one of the speaker/microphone 306, the display/touchpad 310, or the peripherals 318 of the computing devices 112, 124.

As noted above, the efficacy of a long-term anti-IL5 mAb or anti-IL5 receptor mAb treatment regimen may not be apparent until the patient 102 has undergone treatment for an extended period of time. In the case of reslizumab, for example, efficacy of the long-term treatment may ordinarily be assessed after the patient 102 has received the drug therapy over a 52 week period. The assessment may conclude with the patient 102 being classified into one of three categories, such as responder, non-responder or indeterminate. Exemplary definitions of a responder or non-responder at 52 weeks are outlined in Table 1 below.

TABLE 1
Definitions of Responder and Non-responder at 52 Weeks

| Non-responder | Responder |
|---|---|
| ≧2 CAE at 52 weeks UNLESS | 0 or 1 CAE at 52 weeks AND |
| at least 1 of the 3 following | at least 1 of the 3 following |
| are met: | are met: |
| >10% change in FEV₁ AND ACQ6 ≦−0.5 | >10% change in FEV₁ |
| at 52 weeks | at 52 weeks |
| >10% FEV₁ AND AQLQ ≧0.5 at 52 | ACQ6 ≦−0.5 at 52 weeks |
| weeks | |
| 50% reduction from historical number of | AQLQ ≧0.5 at 52 weeks |
| CAEs at 52 weeks | |

The foregoing composite definition for response at 52 weeks may be conservative in that it may include not only a reduction in CAEs but may also include improvement on at least one measure of impairment (e.g., FEV₁, ACQ6+, and/or AQLQ+). If the patient 102 experienced two or more CAEs at 52 weeks, the patient 102 may be deemed a non-responder unless the patient 102 showed clinically significant improvement on at least two measures of impairment and/or a 50% reduction from the historic number of CAEs in a 52-week period before treatment commenced. Patients who do not meet the definition of a non-responder or a responder at 52 weeks may be categorized as indeterminate.

While the foregoing definitions may be more conservative than the definition of response in a clinical study, they may be clinically relevant to the healthcare professional 132 and/or the patient 102. Clinically significant changes in ACQ and AQLQ have been based on published MCIDs, as described in Juniper E F, Guyatt G H, Willan A, Griffith L E, Determining a minimal important change in a disease-specific quality of life questionnaire, J Clin Epidemiol 1994; 47(1):81 7 and in Juniper E F, O'Byrne P M, Guyatt G H, Ferrie P J, King D R, Development and validation of a questionnaire to measure asthma control, Eur Respir J 1999; 14(4):902 7. There may not be an established MCID for FEV₁. However, a 10% change in FEV₁ with reslizumab when added on to GINA step 4/5 therapy may be considered clinically significant, as described in Reddel H K, Taylor D R, Bateman E D, Boulet L P, Boushey H A, Busse W W, et al., An official American Thoracic Society/European Respiratory Society statement: asthma control and exacerbations: standardizing endpoints for clinical asthma trials and clinical practice, Am J Respir Crit Care Med 2009; 180(1):59-99.

The disclosed therapy software module may employ an algorithm to predict the probability of the patient 102 being classified as a responder or non-responder in a long-term anti-IL5 mAb or anti-IL5 receptor mAb treatment regimen using clinical parameters processed from the clinical data collected from an initial treatment period. Early identification of responders and non-responders may reduce continued exposure of non-responding patients to unnecessary medication, decreasing the risk of adverse reactions and reducing the cost burden to patients, insurers, healthcare providers, and/or benefit managers. As such, the therapy software module may provide an early indication of anti-IL5 mAb or anti-IL5 receptor mAb response that can support clinical decisions regarding continued therapy on an individual patient basis.

The algorithm within the therapy software module may employ a multinomial logistic regression to predict the probability of the patient 102 falling into one of the three categorical outcomes (e.g., responder, non-responder or indeterminate) at 52 weeks after an initial treatment period lasting less than 52 weeks. In one embodiment, the algorithm may utilize clinical data based on an initial treatment period of 16 weeks.

In one embodiment, the clinical data may include pre-treatment or baseline (BL) metrics, such as:
an ACQ6 score (ACQ6_{BL});
an AQLQ score (AQLQ_{BL});
an FEV₁ measurement (FEV_{1(BL)}); and/or
a number of CAEs in the previous 12 months (CAEs_{BL}).

The clinical data may further include post-initial treatment metrics, such as:
an ACQ6 score (ACQ6₁₆);
an AQLQ score (AQLQ₁₆);
an FEV₁ measurement (FEV₁₍₁₆₎);
and/or the number of CAEs over the initial treatment period (CAEs₁₆).

The clinical data may be used to derive certain clinical parameters, which may include:
a dichotomized change in ACQ6 from the baseline through the initial treatment (X₁);
a dichotomized change in AQLQ from the baseline through the initial treatment (X₂);
a number of CAEs in the year before beginning the initial treatment (X₃);
a quotient of FEV₁ (X₄); and
a number of CAEs during the initial treatment (X₅).

The dichotomized change in ACQ6 from the baseline through the initial treatment (X₁) may represent a difference between the ACQ₁₆ and the ACQ_{BL}. If ACQ₁₆−ACQ_{BL} is less than or equal to (≦)−0.5, then X₁ may equal 1. If ACQ₁₆−ACQ_{BL} is greater than (≧)−0.5, then X₁ may be 0.

The dichotomized change in AQLQ from the baseline to through initial treatment may represent a difference between the AQLQ₁₆ and the AQLQ_{BL}. If AQLQ₁₆−AQLQ_{BL} is greater than or equal to (≧) 0.5, then X₂ may equal 1. If ACLQ₁₆−ACLQ_{BL} is less than (<) 0.5, then X₂ may be 0.

The quotient of FEV₁ may comprise FEV₁₍₁₆₎/FEV_{1(BL)}.

Two linear scores, L1 and L2, may be derived from the foregoing clinical parameters as follows:

L₁=ln(P₁/P₃)=b₀₁+b₁₁X₁+b₂₁X₂+b₃₁X₃+b₄₁X₄+b₅₁X₅ (I)

L₂=ln(P₂/P₃)=b₀₂+b₁₂X₁+b₂₂X₂+b₃₂X₃+b₄₂X₄+b₅₂X₅ (II)

The “b” values (i.e., b₀₁ to b₅₂) in the equations I and II may represent regression coefficients, which may be considered as the weight of each explanatory variable (“X”). Exemplary regression coefficients (“b” values) are shown in Table 2.

TABLE 2
Regression coefficients

| Regression | | Regression | |
|---|---|---|---|
| Coefficient | Value | Coefficient | Value |
| b₀₁ | 0.4366 | b₀₂ | 0.1200 |
| b₁₁ | 0.3363 | b₁₂ | −0.8349 |
| b₂₁ | −0.5302 | b₂₂ | −0.4716 |
| b₃₁ | −0.0049 | b₃₂ | 0.0294 |
| b₄₁ | −2.9191 | b₄₂ | −1.9467 |
| b₅₁ | 2.6337 | b₅₂ | 2.2974 |

With the regression coefficients, L1 and L2 may be expressed as:

L₁=0.4366+0.3363X₁−0.5302X₂−0.0049X₃−2.9191X₄+2.6337X₅ (III)

L₂=0.1200−0.8349X₁−0.4716X₂+0.0294X₃−1.9467X₄+2.2974X₅ (IV)

The linear scores, L1 and L2, may be used to calculate the likelihood or probability (“P”) that the patient 102 will fall into one of the three categorical outcomes:

P₁=(Non-responder)=exp(L1)/(1+exp(L1)+exp(L2)) (V)

P₂=(Indeterminate)=exp(L2)/(1+exp(L1)+exp(L2)) (VI)

P₃=(Responder)=1/(1+exp(L1)+exp(L2)) (VII)

As P₁, P₂, and P₃ may represent the probabilities of the three mutually exclusive outcomes, the sum of the three values may equal 1. In one embodiment, the patient 102 may be predicted to be a responder at 52 weeks to a long-term anti-IL5 mAb or anti-IL5 receptor mAb treatment regimen (e.g., reslizumab) if P₃ is greater than 0.6. The patient 102 may be predicted to be a non-responder if P₂ is less than 0.4 and P₃ is less than 0.6. The probability may be deemed indeterminate if P₂ is greater than 0.4.

As an example, before commencing the treatment regimen, the patient 102 may have the following baseline data points:
ACQ6_{BL}=1.83333;
AQLQ_{BL}=5.875;
CAEs_{BL}=3; and
FEV_{1(BL)}=0.89.

After receiving treatment for 16 weeks, the patient 102 may have the following initial treatment data points:
ACQ6₁₆=0.5;
AQLQ₁₆=6.6875;
CAEs₁₆=0; and
FEV₁₍₁₆₎=1.71

The clinical parameters may be calculated or determined as follows:
X₁=1 given that ACQ6₁₆−ACQ6_{BL}=0.5−1.8333=−1.3333, which is less than (<) 0.5;
X₂=1 given that AQLQ₁₆−AQLQ_{BL}=6.6875−5.875=0.8125, which is greater than (≧) 0.5;
X₃=3, which is the number of CAEs in the previous year before commencing treatment;
X₄=FEV₁₍₁₆₎/FEV_{1(BL)}=1.71/0.89=1.92135; and
X₅=0, which is the number of CAEs in the first 16 weeks of treatment.

Based on the foregoing parameters and the regression coefficients in Table 2, L1 and L2 may be calculated as follows:

L₁=0.4366+0.3363(1)−0.5302(1)−0.0049(3)−2.9191(1.92135)+2.6337(0)=−5.38061; and

L₂=0.1200−0.8349(1)−0.4716(1)+0.0294(3)−1.9467(1.92135)+2.2974(0)=−4.83859.

From L₁ and L₂, the likelihood or probability (“P”) that patient X will be a non-responder (P₁), indeterminate (P₂), or responder (P₃) may be calculated as follows:

P₁=exp(L₁)/(1+exp(L₁)+exp(L₂))=exp(−5.38061)/(1+exp(−5.38061)+exp(−4.83859))=0.00456;

P₂=exp(L₂)/(1+exp(L₁)+exp(L₂))=exp(−4.83859)/(1+exp(−5.38061)+exp(−4.83859))=0.00785; and

P₃=1/(1+exp(L₁)+exp(L₂))=1/(1+exp(−5.38061)+exp(−4.83859))=0.99087.

Because P₃ is greater than 0.6 after the initial treatment period of 16 weeks, the patient 102 may be classified as a likely responder to a reslizumab treatment, i.e., a patient who would fall under the definition of a responder after receiving approximately 52 weeks of treatment.

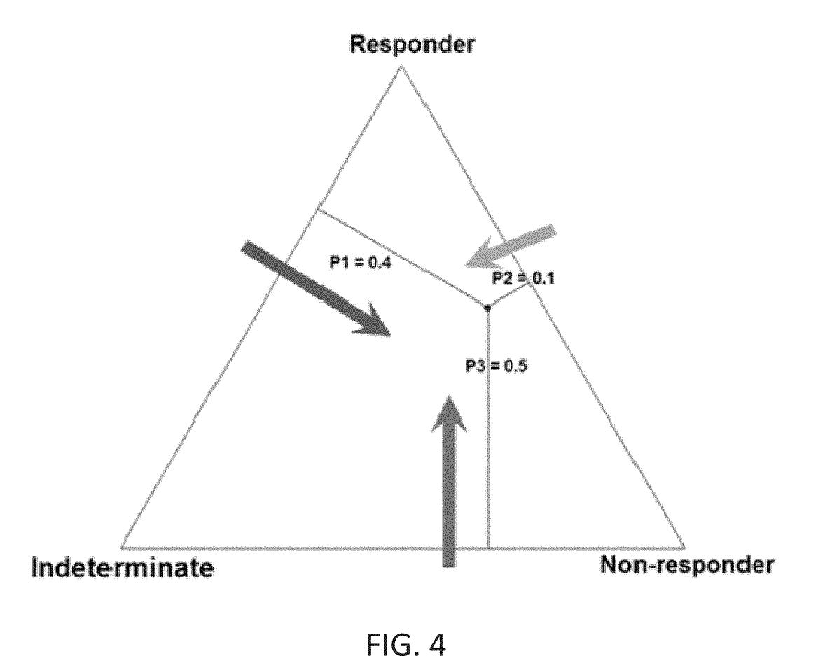

FIG. 4 depicts a two-dimensional geometrical representation of three example categorical probability values for the patient 102. As shown, the three probability values may be represented by the vertices of an equilateral triangle. In particular, a P₃ of 1 may be seen as a data point at the apex of the triangle, representing a likely responder. Alternatively, a P₁ of 1 may be depicted as a data point at the lower right vertex of the triangle, representing a likely non-responder. Lastly, a P₂ of 1 may be depicted as a data point at the lower left vertex of the triangle, which may be labeled as indeterminate.

In the specific example shown in FIG. 4, the therapy software module determined the three categorical probability values to be P₁=0.4, P₂=0.1, and P₃=0.5 based on an exemplary set of clinical parameters. The numerical values were plotted as the distance from the side of the triangle along a perpendicular line running through the opposite vertex. Thus, the value of P₁=0.4 was measured from the left side of the triangle toward the “non-responder” vertex in the lower right, as P₁ is the probability that the patient 102 will be a non-responder. In this example, since P₃<0.6 and P₂<0.4, the patient 102 may be predicted to be a non-responder at 52 weeks based on the clinical parameters obtained after the initial treatment period, which may be 16 weeks.

The foregoing exemplary algorithm was derived using the 16-week data from patients who participated in two Phase 3, randomized, double-blind studies (Studies 3082 and 3083), which are described in U.S. patent application Ser. No. 14/838,503 (published as U.S. App. Pub. No. US2016-0102144) and Int'l Patent App. No. PCT/US2015/047357 (published as Int'l Pub. No. WO2016/040007), respectively, each of which are incorporated herein in their entirety. Studies 3082 and 3083 represented 2 of the 3 adequate and well-controlled studies that served as the basis for the Committee for Medicinal Products for Human Use's (CHMP) positive opinion of reslizumab in the treatment of severe eosinophilic asthma inadequately controlled on high-dose inhaled corticosteroids (ICS) with another medicinal product for maintenance treatment. The studies were identical in design (reslizumab 3 mg/kg every 4 weeks for 52 weeks) with a primary endpoint of frequency of CAEs during the 52-week treatment period. A third study (Study 1 from US2016-0102144 and WO2016/040007) was only 16 weeks in duration and, as such, was not used to model the outcome at 52 weeks.

At baseline, patients had screening eosinophil counts of ≧400 cells/pi blood and had asthma that was uncontrolled on ICS-based therapy (80% on ICS plus another medicinal product for maintenance treatment) as evidenced by an ACQ7 score of 2.65 and an average of approximately 2 CAEs over the previous 12 months. Secondary endpoints of relevance to the model included change from the FEV_{1(BL)} at 16 weeks, change from the AQLQ_{BL} at 16 weeks, and change from the ACQ_{BL} at 16 weeks. The primary efficacy endpoint was met in both studies. Reslizumab 3.0 mg/kg significantly reduced the frequency of CAEs over 52 weeks compared with placebo (p<0.0001) in Studies 3082 and 3083 by 50% and 59%, respectively. Reslizumab also showed significant treatment benefits on lung function (FEV₁), the ACQ, and the AQLQ in both studies.

The European Medicines Agency proposed that reslizumab be used in the adult population with severe eosinophilic asthma inadequately controlled despite high-dose ICS plus another medicinal product for maintenance treatment as defined by meeting Global Initiative for Asthma (GINA) 4 or GINA 5 criteria (as described in Global Initiative for Asthma (GINA) REPORT, 2014). Consistent with this recommendation, the population that served as the data source for the model included adult (≧18 years old) patients meeting GINA 4 or 5 criteria who were treated with reslizumab in Studies 3082 and 3083. Among the 383 patients meeting these criteria, 321 patients used in constructing the algorithm were those for whom data were available for all assessment points.

In the algorithm, data collected from the patients after 16 weeks of treatment may be used to predict how an individual patient might respond after 52 weeks of treatment. The reference point of 16 weeks may be selected for the algorithm as indicative of early improvement because it may represent the time point by which improvements in asthma impairment, as measured by FEV₁, the ACQ, and the AQLQ, may be expected to have plateaued in most patients based on the results of the Phase 3 studies. Furthermore, the first AQLQ assessment may be performed at 16 weeks to allow quality of life to be factored into the model. Patients received 4 doses of reslizumab 3 mg/kg by 16 weeks.

The results of the algorithm/model are displayed in Table 3 below. Based on 16 weeks of treatment from the studies, the algorithm predicted 276 patients (86%) as responders and 26 patients (8%) as non-responders. On the actual 52 week outcome, the algorithm had correctly predicted 248 patients (90%) of the predicted responders and 13 patients (50%) of the predicted non-responders. There were also 19 patients (6%) with an indeterminate outcome at 16 weeks. The sensitivity and specificity of the algorithm were determined excluding the patients who were categorized as indeterminate. The algorithm had a sensitivity of 98.02% (95% CI: 95.45%, 99.36%) (i.e., correctly predicted 248 of the 260 actual responders), indicating that it was successful in predicting responders. The specificity of the model was 54.17% (95% CI: 32.82%, 74.45%) (i.e., correctly predicted 13 of the 32 actual non-responders). The positive predictive value was 95.75% (95% CI: 92.53%, 97.86%), and the negative predictive value was 72.22% (95% CI: 46.52%, 90.31%). The large CI around the estimated negative predictive value is reflective of the relatively small numbers of actual non-responders in the population (<5%).

Lin's concordance correlation coefficient (as described in Lin Li, A concordance correlation coefficient to evaluate reproducibility, Biometrics 1989; 45(1):255-68) measures the overall agreement between actual responder/indeterminate/non-responder status at 52 weeks versus the predicted status using the 16-week data to evaluate reproducibility and inter-subject reliability. In a 25,000 bootstrap sample assuming no relationship between the covariates and dependent variable, Lin's correlation ranged from −0.21 to 0.21. Lin's correlation for the data in this model is 0.56, indicating a highly statistically significant result.

TABLE 3
Comparison of Actual Versus Predicted Response

Table of actual by predicted

predicted

| Frequency | actual | 1 | 2 | 3 | Total |
|---|---|---|---|---|---|
| | 1 | 13 | 8 | 11 | 32 |
| | 2 | 8 | 4 | 17 | 29 |
| | 3 | 5 | 7 | 248 | 260 |
| | Total | 26 | 19 | 276 | 321 |

1 = Non-responder;
2 = Indeterminate;
3 = Responder

The model was validated using a statistical technique known as jackknife resampling, as described in Efron B, Stein C. (May), The jackknife estimate of variance, The Annals of Statistics 1981; 9(3):586-96. In this approach, the analysis was run “n” times (i.e., 321 times, once for each patient in the model), where each observation was systematically left out of the data one at a time, and the model was fit to the remaining “n−1” (320) observations. The jackknife estimate predicts the patient that was removed from the analysis. The jackknife validation results are shown in Table 4.

TABLE 4
Jackknife Validation of Model: Comparison of Actual Versus Predicted
Response

Table of actual by predicted

predicted

| Frequency | actual | 1 | 2 | 3 | Total |
|---|---|---|---|---|---|
| | 1 | 10 | 12 | 10 | 32 |
| | 2 | 2 | 11 | 16 | 29 |
| | 3 | 22 | 8 | 230 | 260 |
| | Total | 34 | 31 | 256 | 321 |

1 = Non-responder;
2 = Indeterminate;
3 = Responder

In addition to the jackknife validation, a cross-study validation was performed to assess how well the model could be generalized to a separate set of data that was not used in the development of the model. In this model validation method, the results of an analysis performed on 1 dataset (training set) were used to estimate how a predictive model will perform when applied to an independent dataset (validation set). Among the 321 patients used in constructing the full model, 162 patients originated from Study 3082, and 159 patients originated from Study 3083. When Study 3082 was used as the training set and Study 3083 as the validation set and the resulting model applied to Study 3083, the predicted values for the Study 3083 patients were 100% in agreement with the predictions obtained using the full model. When Study 3083 was used as the training set and Study 3082 as the validation set, the predicted values for the Study 3082 patients were 93.7% (149/159) in agreement with the predictions obtained using the full model. For the 10 patients in Study 3083 where the predictions from the cross-study validation differed from the full data prediction, 7 were predicted as non-responders in the full data analysis and indeterminate in the validation, 1 was predicted as a responder in the full data analysis and indeterminate in the validation, and 2 were predicted as responders in the full data analysis and non-responders in the validation.

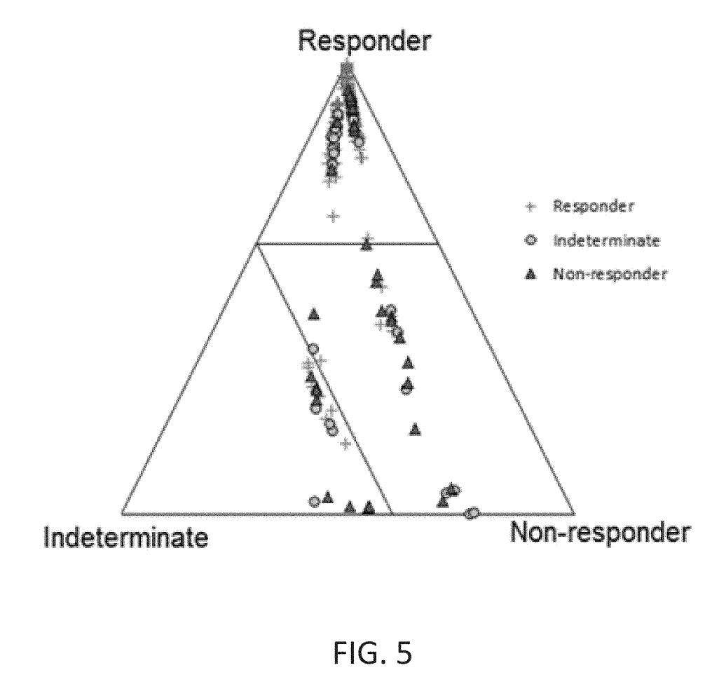

FIG. 5 is a plot showing the distribution of the trichotomous probability data points for each of the 321 patients from Studies 3082 and 3083 along with the actual response determinations made at 52 weeks (which is also summarized in Table 3 above). More specifically, based on the baseline and initial treatment clinical parameters of the 321 patients after 16 weeks, the algorithm identified 276 patients as likely responders, 26 as likely non-responders, and 19 as indeterminate. Of the 276 patients identified as likely responders, 248 were ultimately classified as responders at 52 weeks while 11 were labeled as non-responders and 17 as indeterminate. Of the 26 patients identified as likely non-responders by the algorithm, 13 were ultimately classified as non-responders at 52 weeks while 5 were labeled as responders and 8 as indeterminate. Of the 19 patients identified as indeterminate, 4 were ultimately classified as indeterminate at 52 weeks while 7 were labeled as responders and 8 as non-responders.

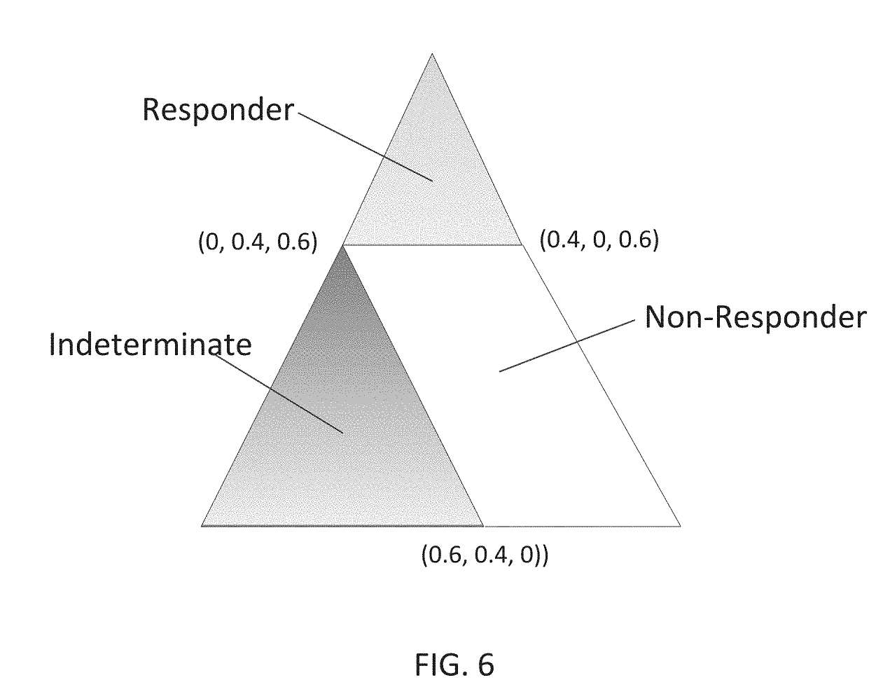

FIG. 6 illustrates the geometric demarcation of the three categorical outcomes depicted in FIGS. 4 and 5, i.e., responder, non-responder and indeterminate.

What is claimed:

1. A system for managing a long-term anti-IL5 mAb or anti-IL5 receptor mAb treatment regimen for a patient suffering from severe asthma, the system comprising:
an inhaler having a pressure sensor and a wireless transceiver, wherein the pressure sensor is configured to sense first and second inhalations by the patient from the inhaler and the wireless transceiver is configured to wirelessly transmit the first and second sensed inhalations; and
a computing device configured to process baseline clinical data and initial clinical data, the baseline clinical data including data gathered prior to the commencement of an anti-IL5 mAb or anti-IL5 receptor mAb initial treatment period and the initial clinical data including data gathered following the initial treatment period,
wherein as part of the processing of the baseline clinical data the computing device is configured to:
present a first asthma control questionnaire (ACQ) and a first asthma quality of life questionnaire (AQLQ);
compute a baseline ACQ score (ACQ_{BL}) and a baseline AQLQ score (AQLQ_{BL}) based on the patient's responses to the first ACQ and the first AQLQ;
store a baseline number of clinical asthma exacerbations experienced by the patient (CAE_{BL});
receive the first sensed inhalation from the inhaler; and
determine a baseline lung function metric based on the first sensed inhalation;
wherein as part of the processing of the initial clinical data the computing device is configured to:
present a second ACQ and a second AQLQ;
compute an initial treatment ACQ score (ACQ_{IT}) and an initial treatment AQLQ score (AQLQ_{IT}) based on the patient's responses to the second ACQ and the second AQLQ;
store a number of clinical asthma exacerbations experienced by the patient over the initial treatment period (CAE_{IT});
receive the second sensed inhalation from the inhaler; and
determine an initial treatment lung function metric based on the second sensed inhalation;
wherein the computing device is further configured to compute clinical parameters based on the baseline clinical data and the initial clinical data,
wherein as part of computing the clinical parameters the computing device is configured to:
calculate a change between the ACQ_{BL} and the ACQ_{IT};
calculate a change between the AQLQ_{BL} and the AQLQ_{IT}; and
calculate a quotient of the initial treatment lung function metric over the baseline lung function metric;
wherein the computing device is further configured to compute a probability of the patient being responsive to the long-term treatment regimen based on (1) the change between the ACQ_{BL} and the ACQ_{IT}, (2) the change between the AQLQ_{BL} and the AQLQ_{IT}, (3) the quotient of the initial treatment lung function metric over the baseline lung function metric, (4) the CAE_{BL}, and (5) the CAE_{IT}, and
wherein the computing device is configured to generate a notification of the computed probability.

2. The system of claim 1, wherein the initial treatment period is approximately 16 weeks, and wherein the long-term treatment regimen comprises a period of approximately 52 weeks.

3. The system of claim 1, wherein reslizumab is administered during the initial treatment period.

4. The system of claim 3, wherein reslizumab is administered beyond the initial treatment period if the computed probability indicates that the patient is classified as a likely responder to the long-term treatment regimen.

5. The system of claim 3, wherein the administration of reslizumab is discontinued after the initial treatment period if the computed probability indicates that the patient is classified as a likely non-responder to the long-term treatment regimen.

6. The system of claim 1, wherein the computed probability indicates whether the patient is classified as a responder, non-responder or indeterminate.

7. The system of claim 1, wherein the computing device is configured to compute the probability by calculating a first linear score (L1) and a second linear score (L2),
wherein L1 equals b₀₁+b₁₁X₁+b₂₁X₂+b₃₁X₃+b₄₁X₄+b₅₁X₅,
wherein L2 equals b₀₂+b₁₂X₁+b₂₂X₂+b₃₂X₃+b₄₂X₄+b₅₂X₅,
wherein X₁ equals the change between the ACQ_{BL} and the ACQ_{IT};
wherein X₂ equals the change between the AQLQ_{BL} and the AQLQ_{IT};
wherein X₃ equals CAE_{BL};
wherein X₄ equals the quotient of the initial treatment lung function metric over the baseline lung function metric;
wherein X5 equals CAE_{IT}; and
wherein b₀₁ to b₅₂ comprise regression coefficients for X₁ to X₅.

8. The system of claim 7, wherein the computing device is further configured to compute the probability by calculating a non-responder probability value (P₁), an indeterminate probability value (P₂), and a responder probability value (P₃),
wherein P₁ equals exp(L1)/(1+exp(L1)+exp(L2));
wherein P₂ equals exp(L2)/(1+exp(L1)+exp(L2)); and
wherein P₃ equals 1/(1+exp(L1)+exp(L2)).

9. The system of claim 8, wherein the computing device is configured to indicate that the patient is a likely responder to the long-term treatment regimen if P₃ is greater than 0.6.

10. The system of claim 8, wherein the computing device is configured to indicate that the patient is a likely non-responder to the long-term treatment regimen if P₂ is less than 0.4 and P₃ is less than 0.6.

11. The system of claim 8, wherein the computing device is configured to indicate that the computed probability is indeterminate if P₂ is greater than 0.4.

12. A method of managing a long-term anti-IL5 mAb or anti-IL5 receptor mAb treatment regimen for a patient suffering from severe asthma, the method comprising:
processing baseline clinical data prior to commencing an initial treatment period, wherein the processing of baseline clinical data includes:
presenting, via a display, a first asthma control questionnaire (ACQ) and a first asthma quality of life questionnaire (AQLQ);
computing a baseline ACQ score (ACQ_{BL}) and a baseline AQLQ score (AQLQ_{BL}) based on responses to the first ACQ and the first AQLQ;
storing a baseline number of clinical asthma exacerbations experienced by the patient (CAE_{BL});
sensing a first inhalation via an inhaler having a pressure sensor; and
determining a baseline lung function metric based on the first sensed inhalation;
processing initial clinical data associated with an initial treatment period, wherein the processing of initial clinical data includes:
presenting, via the display, a second ACQ and a second AQLQ;
computing an initial treatment ACQ score (ACQ_{IT}) and an initial treatment AQLQ score (AQLQ_{IT}) based on responses to the second ACQ and the second AQLQ;
storing a number of clinical asthma exacerbations experienced by the patient over the initial treatment period (CAE_{IT});
sensing a second inhalation via the inhaler; and
determining an initial treatment lung function metric based on the second sensed inhalation;
computing clinical parameters based on the baseline clinical data and the initial clinical data, wherein the processing of the clinical parameters includes:
computing a change between the ACQ_{BL} and the ACQ_{IT};
computing a change between the AQLQ_{BL} and the AQLQ_{IT}; and
computing a quotient of the initial treatment lung function metric over the baseline lung function metric;
computing a probability of the patient being responsive to the long-term treatment regimen based on (1) the change between the ACQ_{BL} and the ACQ_{IT}, (2) the change between the AQLQ_{BL} and the AQLQ_{IT}, (3) the quotient of the initial treatment lung function metric over the baseline lung function metric, (4) the CAE_{BL}, and (5) the CAE_{IT}; and
generating a notification of the computed probability.
